(19) United States
(12) Plant Patent Application Publication
(10) Pub. No.: US 2008/0134387 P1
(43) Pub. Date: Jun. 5, 2008

(54) Coreopsis plant named 'RP #4'

(75) Inventor: Harini Korlipara, Canby, OR (US)

Correspondence Address: KLARQUIST SPARKMAN, LLP, 121 SW SALMON STREET, SUITE 1600, PORTLAND, OR, 97204, US

(73) Assignees: Terra Nova Nurseries, Inc.; The Ivy Farm

(21) Appl. No.: 11/592,083

(22) Filed: Nov. 1, 2006

(51) Int. Cl. A01H 5/00

(52) U.S. Cl. PLT/263.1

(57) ABSTRACT

A new and distinct Coreopsis plant named ‘RP #4’ characterized by very free flowering rusty yellow colored, daisy-like flowers, a low mounding habit, and excellent vigor.

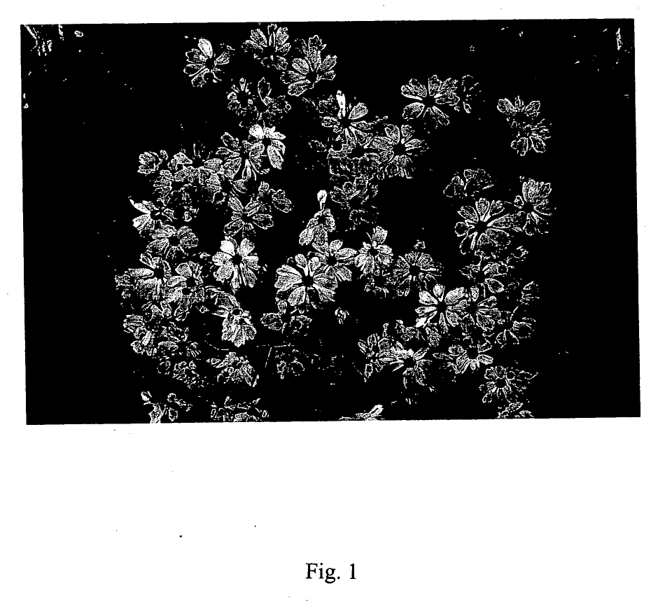

### BOTANICAL DESIGNATION

Coreopsis hybrid (of unknown origin)

### VARIETY DENOMINATION

‘RP #4’

### CROSS REFERENCE TO

co-pending applications for Coreopsis ‘Rum Punch’ (U.S. Plant patent application Ser. No. 11/488,815), Coreopsis ‘RP #1’ (co-pending application), Coreopsis ‘RP #5’ (co-pending application)

### BACKGROUND OF THE INVENTION

The present invention relates to a new and distinct plant of Coreopsis and given the cultivar name ‘RP #4’. Coreopsis is in the family Asteraceae. This new cultivar originated as a tissue culture mutation from Coreopsis ‘Rum Punch’ (U.S. Plant patent application Ser. No. 11/488,815). ‘RP #4’ was one of several interesting mutations found from tissue cultured plants.

### SUMMARY OF THE INVENTION

The following traits have been repeatedly observed and are determined to be the unique characteristics of ‘RP #4’. These characteristics in combination distinguish ‘RP #4’ as a new and distinct cultivar:
1. Unique rusty-yellow daisy-like flowers.
2. Low, mounding habit.
3. Very free flowering.
4. Excellent vigor.

This new cultivar has been reproduced only by asexual propagation (cuttings and tissue culture). Each of the progeny exhibits identical characteristics to the original plant. Asexual propagation by cuttings and tissue culture using standard micropropagation techniques with terminal and lateral shoots, as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE PHOTOGRAPH

The photograph shows a one year old Coreopsis ‘RP #4’ growing in the ground in the garden in full sun in August in Canby, Oreg.

### DETAILED PLANT DESCRIPTION

The following is a detailed description of the new Coreopsis cultivar based on observations of a one-year-old specimen grown in the ground in full sun under typical outdoor conditions in the trial fields in August in Canby, Oreg. Canby is Zone 8 on the USDA Hardiness map. Temperatures range from a high of 95 degrees F in August to an average of 32 degrees F in January. Normal rainfall in Canby is 42.8 inches per year. The color descriptions are all based on The Royal Horticultural Society Colour Chart.
Plant:
   Type.—herbaceous perennial, grown as an annual.
   Hardiness.—USDA Zone 9-10.
   Size.—45 cm wide and 30 cm tall to top of flowers.
   Form.—low mound with freely branching stems.
   Vigor.—excellent.
   Roots.—fibrous, freely branching, fine, and Yellow White 158A; roots develop easily from stem cuttings.
Stem:
   Type.—ascending.
   Size.—30 cm tall and 1 to 4 mm wide.
   Internode length.—1.5 to 5.5 cm.
   Surface.—glabrous.
   Color.—Green 147B.
Leaf:
   Type.—simple.
   Shape.—mostly linear with some basal leaves with one to two linear side lobes, side lobe(s) perpendicular to main lobe, reflexed.
   Arrangement.—opposite.
   Size.—grows to 5.5 cm long and 2.5 mm wide when linear, side lobe(s) from near base of leaf, grow to 22 mm long and 1 mm wide.
   Margins.—entire.
   Apex.—acute.
   Base.—clasping.
   Surface texture.—glabrous on both sides.
   Venation.—pinnate.
   Color.—Top side — Green 147A Bottom — Green 148B.
Immature inflorescence: Globular, 6 mm wide and 6 mm deep, Greyed Purple 185A on tip and seams with Greyed Orange 175A in the middle and the bottom Yellow Green 147D
Inflorescence:
   Type.—long stalked terminal heads of daisy type flowers.
   Peduncle.—grows to 8.5 cm tall and 0.5 mm wide, glabrous, Green 137A.
   Size.—3.5 cm wide and 8 mm deep.
   Ray florets.—8 in number, no pistil or stamen, obovate, the tip is three to four lobed with the terminal 2 lobes the longest, margins entire, base cuneate, grows to 17 mm long and 9 mm wide, glabrous on both surfaces, 3 mm claw at base, laminae with linear appendage (1 or 2) on side and/or opposite and upright, 10 mm long.
   Color.—Ray floret, topside — somewhat variable, sometimes Greyed Orange 167A darkening to 172B near the tip, others are Greyed Orange 163A; both with Greyed Orange 163B at base; older flowers turn mustard yellow, Greyed Orange 163B overall Ray floret, bottom side — Yellow 11B with blotches of Greyed Orange 176A near the tips on some of the ray florets.
   Cone.—conic in shape, grows to 6 mm wide and 3 mm deep, Greyed Purple 187A.
   Disc florets.—Corolla — 3 mm long and less than 1 mm wide, tubular, 4 lobed, Greyed Purple 187A on the lobes to Yellow 11A on tube inside and out, glabrous Pistil — 5 mm long, 2-branched stigma extruding from the corolla, style 3.5 mm long, Orange 17A overall, ovary 1.5 mm long, White 155A Stamen — 4, filaments 3 mm long, extruding, Black 202A, pollen Orange 17A.
   Phyllaries.—in 2 series, series closet to ray florets 8 in number, ovate, entire, acuminate, lobes 6 mm long and 3 mm wide, glabrous, Greyed Orange 167A on top half and Yellow Green 147A on bottom half; lower series 8 in number, lobes 2 mm long and 1.5 mm wide at the base, ovate, entire, acute, glabrous, Yellow Green 147A.
   Bloom period.—June through September in Canby, Oreg.
   Fragrance.—light, daisy like.
Seed: None produced
   Fertility.—Infertile.
Disease and pests: Coreopsis are susceptible to mildew and fungal spots. No known resistances on the new cultivar.

### COMPARISONS TO SIMILAR COREOPSIS

Compared to Coreopsis ‘Limerock Ruby’ (US Plant Patent Application No. 20030066114), this new cultivar has a new and unique flower color of rusty yellow rather than rose pink.

Compared to Coreopsis ‘Rum Punch’ (U.S. Plant patent application Ser. No. 11/488,815), the new cultivar has flowers of a rusty yellow color rather than orangey rose.

Compared to Coreopsis ‘RP #5’, a co-pending application, Coreopsis ‘RP #4’ has rusty yellow flower color rather than rust orange.

I claim:

1. A new and distinct Coreopsis plant as herein illustrated and described.
